(19) United States
(12) Patent Application Publication
(10) Pub. No.: US 2007/0119451 A1
(43) Pub. Date: May 31, 2007

(54) Nasal device for delivery to the olfactory region

(76) Inventors: Jingli Wang, Mountain View, CA (US); Johanna H. Bentz, Newark, CA (US); Rolfe Anderson, Saratoga, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/606,244

(22) Filed: Nov. 29, 2006

Related U.S. Application Data
(60) Provisional application No. 60/740,488, filed on Nov. 29, 2005.

(51) Int. Cl. A61M 5/178; A61M 15/00; A61M 16/00

(52) U.S. Cl. 128/203.15; 128/207.18; 604/37; 128/203.28

(57) ABSTRACT

A device for delivery of a substance to the olfactory region is described. The device includes a nosepiece and an elongate tubular member slidably disposed within the nosepiece for movement between a retracted position and an extended position. The tubular member is in flow communication with a reservoir containing the substance to be delivered. During use, the tubular member extends from the device, to direct the substance toward the olfactory region.

### CROSS REFERENCE TO RELATED U.S. APPLICATION DATA

The present application claims the benefit of provisional application 60/740,488 filed Nov. 29, 2005, of which is incorporated by reference herein.

### TECHNICAL FIELD

The subject matter described herein relates to a nasal delivery device designed for delivering a substance to the olfactory region of a subject.

### BACKGROUND

There are many advantages to intranasal administration of medications and other compositions that include, among others, a direct route to the blood stream, avoidance of hepatic first pass metabolism, higher bioavailability, ease and convenience, and proximity to the central nervous system. Normally, delivery of substances to the central nervous system is difficult, due to the blood brain barrier. The blood-brain barrier arises primarily from the endothelium of the brain capillaries, through which few molecules can pass. The blood-brain barrier has prevented the delivery of therapeutic drugs for treating Alzheimer's, Parkinson's, stroke, spinal cord injury, depression, and other central nervous system disorders.

Involved in sensing odors and chemicals, the olfactory region inside the nasal cavity provides a unique and direct connection between the brain and the external environment. A number of studies reported drugs that do not or poorly cross the blood-brain barrier can be rapidly delivered to the CNS when applied inside the nasal cavity. While intranasal delivery specifically to the olfactory region potentially provides a route for delivery of agents to the central nervous system, the olfactory region is difficult to access using conventional nasal delivery devices. The olfactory region is located in the uppermost portion of the nasal cavity, where less than 10% of the inhaled air flows. Conventional nasal sprays deposit the majority of the dosage in the lower portion of the nasal cavity, with very little reaching the olfactory region. Devices that provided targeted delivery to the olfactory region are needed in the art.

The foregoing examples of the related art and limitations related therewith are intended to be illustrative and not exclusive. Other limitations of the related art will become apparent to those of skill in the art upon a reading of the specification and a study of the drawings.

### SUMMARY

The following aspects and embodiments thereof described and illustrated below are meant to be exemplary and illustrative, not limiting in scope.

In one aspect, a device for delivery of a substance to the olfactory regions is provided. In one embodiment, the device includes a nosepiece adapted to form a substantially gas-tight seal with a nostril, and an elongate tubular member slidably disposed within the nosepiece. The tubular member is adapted for movement between a retracted position and an extended position. The tubular member has an outlet at a distal end, and, at proximal end, a structure suitable for retaining the tubular member within the nosepiece when the member is in its extended position. The tubular member also comprises a delivery channel that extends from the distal end to the proximal end, the tubular member being adapted for movement in response to fluid flow from the proximal to the distal ends. The device also includes a reservoir in flow communication with said elongate tubular member, said reservoir containing the substance to be delivered.

In other aspects, methods of using the device and of delivery substances for therapy are described.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1B shows the human nasal cavity with a device according to one embodiment positioned in the nasal vestibule prior to actuation (FIG. 1A) and after actuation (FIG. 1B);

FIGS. 2A-2C are cross-sectional views of the device of FIGS. 1A-1B, with the elongate member fully retracted (FIG. 2A), partially extended (FIG. 2B) and fully extended (FIG. 2C);

FIGS. 3A-3C are illustrations of a device according to another embodiment;

FIGS. 4A-4B are illustrations of a device according to another embodiment;

FIG. 4C is a cross-sectional view of the device shown in FIGS. 4A-4B;

FIGS. 5A-5C show the human nasal cavity with a device according to another embodiment positioned in the nasal vestibule prior to actuation (FIG. 5A) and after actuation (FIGS. 5B-5C);

FIGS. 6A-6B show a prototype device before (FIG. 6A) and after (FIG. 6B) actuation;

FIGS. 7A-7B show the prototype device inserted into a model nose (FIG. 7A) and after (FIG. 7B) actuation;

FIG. 7C shows a model nasal cavity;

FIGS. 8A-8D show another prototype device before (FIGS. 8A-8B) and after (FIG. 8C-8D) actuation;

FIGS. 9A-9C show the liquid spray distribution into model nasal cavities after delivery of liquid from a commercially available 12-hour decongestant spray bottle, the prototype of FIGS. 8A-8D, and a commercially available nebulizer;

FIGS. 10A-10C show the liquid spray distribution into model nasal cavities after delivery of liquid from the prototype of FIGS. 8A-8D (FIG. 10A) and a commercially available multi-dose, non-pressurized device from Valois Corporation (VP6, FIGS. 10B-10C);

FIG. 11 is a bar graph showing the quantitative distribution of liquid spray, in percent total, in various nasal cavity regions achieved by delivery from a commercially-available 12 hour decongestant spray bottle (Nostrilla), multi-dose, non-pressurized devices from Valois Corporation (VP6, VP7), the prototype of FIGS. 8A-8D, and a commercially available nebulizer operated at slow and fast.

### DETAILED DESCRIPTION

I. Nasal Delivery Device and Method of Use

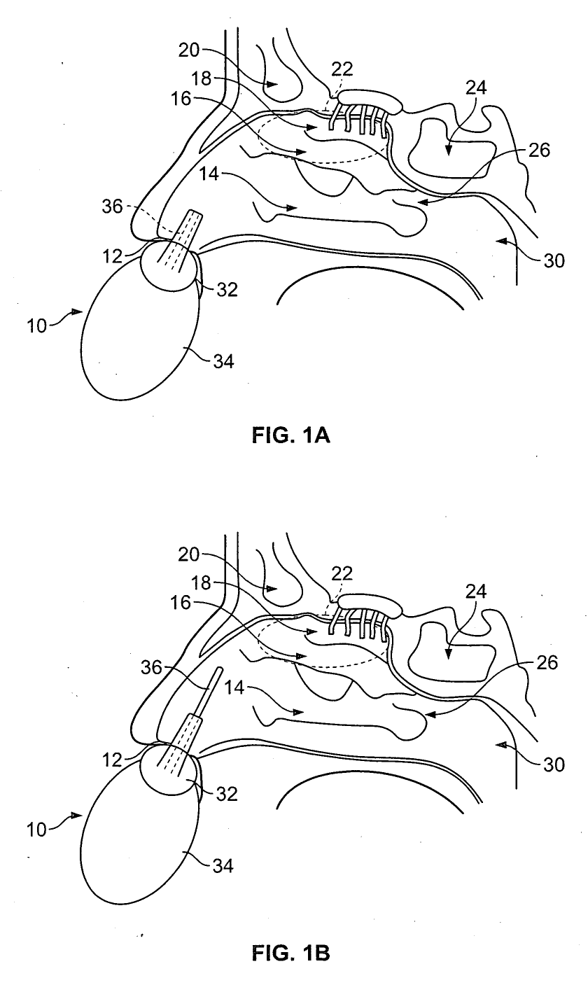

In one embodiment, a device for administering a substance to the olfactory region of a subject is described. Since the olfactory mucosa provides a pathway into the olfactory bulb and into the interconnected areas of the brain and the cerebrospinal fluid, the device is particularly suited for the treatment or prophylaxis of conditions or disorders in the brain or in the central nervous system, or having a therapeutic target in the brain or in the central nervous system. The components of the intranasal delivery device are first described, followed by a description of the device in use. FIGS. 1-8 show particular embodiments of the intranasal delivery device, and are merely examples of devices. Those of skill can appreciate that various changes can be made to the devices described herein while retaining the inventive features, now to be described.

FIGS. 1A-1B illustrate a device in accord with one embodiment, where the device 10 is shown inserted into the nasal vestibule 12 of a subject, with the nasal cavity of the subject shown for purposes of discussion. The nasal vestibule opens into the inferior, middle, and superior conchae, indicated at 14, 16, 18, respectively. The frontal sinus 20 lies forward of and above the conchae. The area enclosed by the dotted line, indicated at 22, indicates the olfactory region. The sphenoid sinus is indicated at 24, with the internal nares indicated at 26. The nasal cavity opens into the nasopharynx 30. Device 10 is comprised of a nosepiece 32 that is preferably formed of a material capable for forming a snug fit with the nostril wall. Connected to the nosepiece by any manner common to the art is a main body 34 in which a substance to be delivered is contained. How the substance is contained in the main body is not critical and may be achieved by placing the substance directly into the main body or may be held within a containing member disposed within the main body. Device 10 also includes an elongate tubular member 36, shown in shadow in FIG. 1A. Tubular member 36 is slidably disposed in the nosepiece, for movement between a retracted position, as seen in FIG. 1A, and an extended position, as shown in FIG. 1B. As will be further described below, when device 10 is inserted into the nasal vestibule the nosepiece extends into the vestibule. Actuation of the device causes the elongate member to move from its retracted position to an extended position in order to direct the active substance toward the olfactory region.

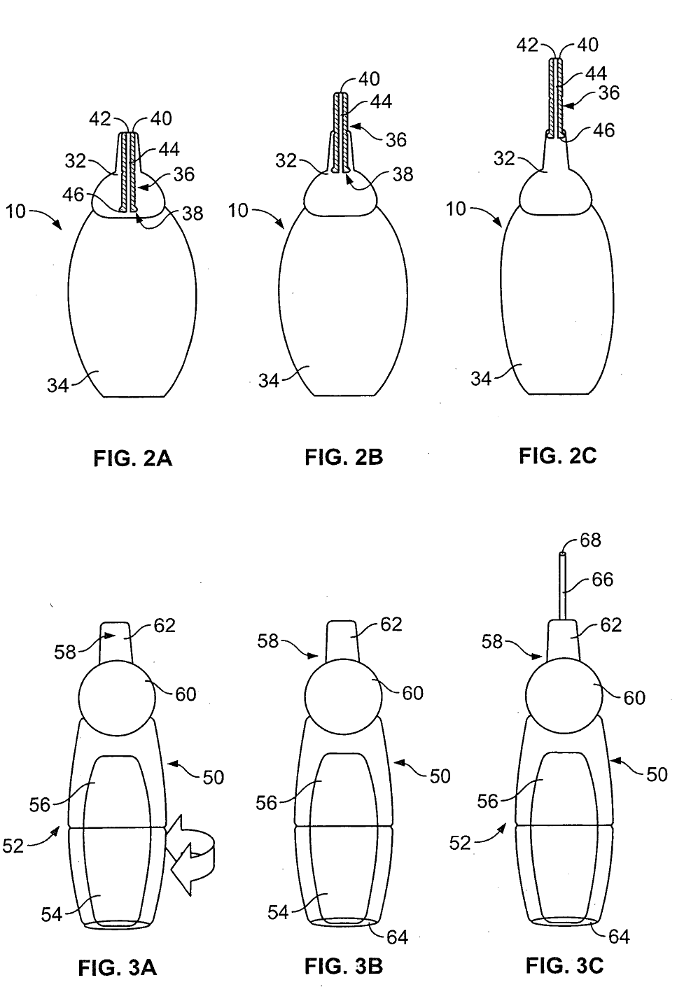

Cross-sectional views of device 10 are shown in FIGS. 2A-2C. Elongate member 36, seen in FIG. 2A in its fully retracted position, includes a proximal end 38 and a distal 40, the distal end having an outlet 42 for release of the active substance to be delivered. In use the substance travels via delivery channel 44 from the body of the device to the outlet. In this embodiment, proximal end 38 includes a detent 46 that serves to retain the elongate member within the nosepiece when the elongate member is in its fully extended position, as seen in FIG. 2C. Actuation of the device, by pressing or squeezing on body 34 in the device shown here, forces air and the substance retained in the main body into the delivery channel and at the same time urges movement of the elongate member from its relaxed position (FIG. 2A) toward its extended position (FIG. 2C). It will be appreciated that delivery of the substance may occur when the elongate member is in a partially extended position, as shown in FIG. 2B.

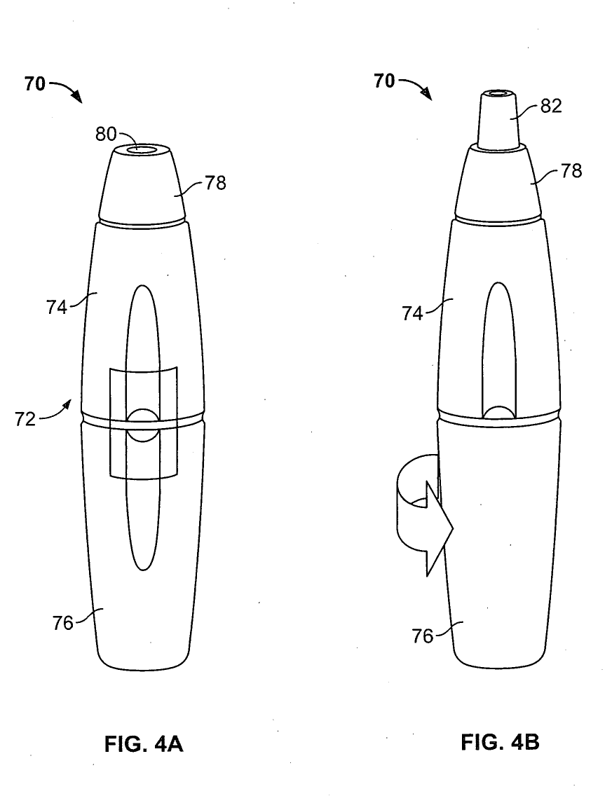

Another embodiment of a device is shown in FIGS. 3A-3C. Device 50 includes a main body 52 having an upper body portion 56 and a moveable lower body portion 54. A nosepiece 58 is connected or integrally formed with the main body, and specifically with the upper body portion. Nosepiece 58 includes a rounded member 60 for fitting against a user's nostril and a nozzle portion 62 that extends into the nasal vestibule. Lower body portion 56 is moveable, as indicated by the arrow in FIG. 3A, clockwise and/or counter-clockwise. Rotation of the lower body portion 180° initiates the device for use, the rotational movement serving to align an internal fluid pathway for intake of air through the bottom of the device, indicated at 64, in response to a user's sniff. FIG. 3B shows the device after the lower body portion has been rotated prior to use. It will be appreciated that the upper body portion, rather than the lower body portion, can be designed for rotational movement. Device 50 also includes a tubular member 66, visible in FIG. 3C, that extends from the nozzle in response to an increasing pressure when a user sniffs or inhales air via through the device. The tubular member includes an outlet 68 at its distal end for release of a substance contained within the body or the nosepiece. Not visible in this drawing is the proximal end of the tubular member, which includes a means for retaining the tubular member in the nozzle when the tubular member is fully extended. For example, such a means can be a lip at the proximal end of the tubular member, the lip being slightly larger than the opening in the device through which the tubular member extends. Such a means can also be a length of the tubular member selected such that a portion of the tubular member remains in the device during use. Such a means can also be a gradual change in diameter of the tubular member, such that the proximal portion of the tubular member is of a larger diameter than the distal portion. It will be appreciated that the nozzle or nosepiece rather than the tubular member could include a means to retain the tubular member within the nozzle.

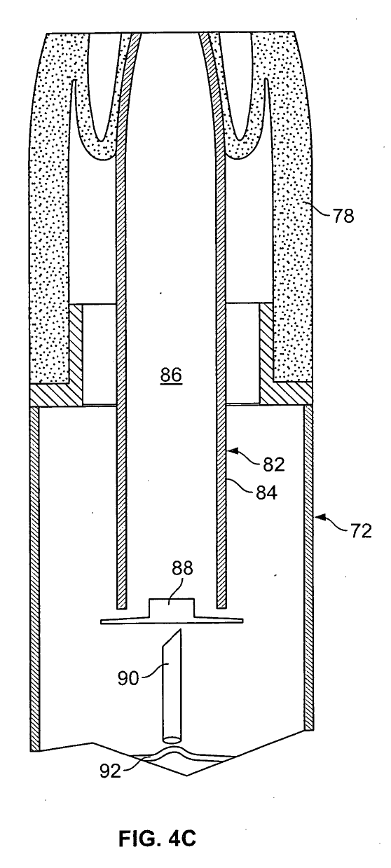

A third device embodiment is shown in FIGS. 4A-4B. Device 70 includes an outer housing 72 having an upper region 74 and a lower, movable region 76. Connected to the upper region of the housing is a rounded head 78, adapted and sized for abutting a nostril opening of a user. Rounded head is adapted for abutting a nostril by, for example, being formed of a compliant material that sealingly engages the nostril opening when placed for use. Head 78 includes an aperture 80 through which a nozzle 82, seen in FIG. 4B, extends upon rotational movement of lower region 76. Nozzle 82 extends into the user's nasal cavity, to direct delivery of the substance contained in the device to the olfactory region. It will be appreciated that the dimensions of nozzle 82 can be varied, for example, to achieve a desired penetration depth in to the nasal cavity.

FIG. 4C is a cross-sectional view of device 70, showing the upper portion of the device. Housing 72 abuts rounded head 78 in which nozzle 82 is disposed. The outer wall 84 of nozzle 82 defines an open channel 86 through which air and a substance to be delivered to a user travel during use of the device. A frangible element 88 is positioned within the outer housing and at a terminal end of the nozzle. Frangible element 88 contains a substance to be delivered to a user of the device. A piercing member 90, depicted as an exemplary needle, is mounted for contact with the frangible element, for example, in response to movement of a curved track 92. Curved track 92 moves in response to rotational movement of the lower housing region, discussed above with respect to FIGS. 4A-4B. As the lower region moves the curved track rotates into position for contact with the piercing member, which punctures the frangible element. Continued rotation of the lower housing region causes the curved track to return to its original position, where contact with the frangible element ceases, and causes the nozzle to extend from the rounded head.

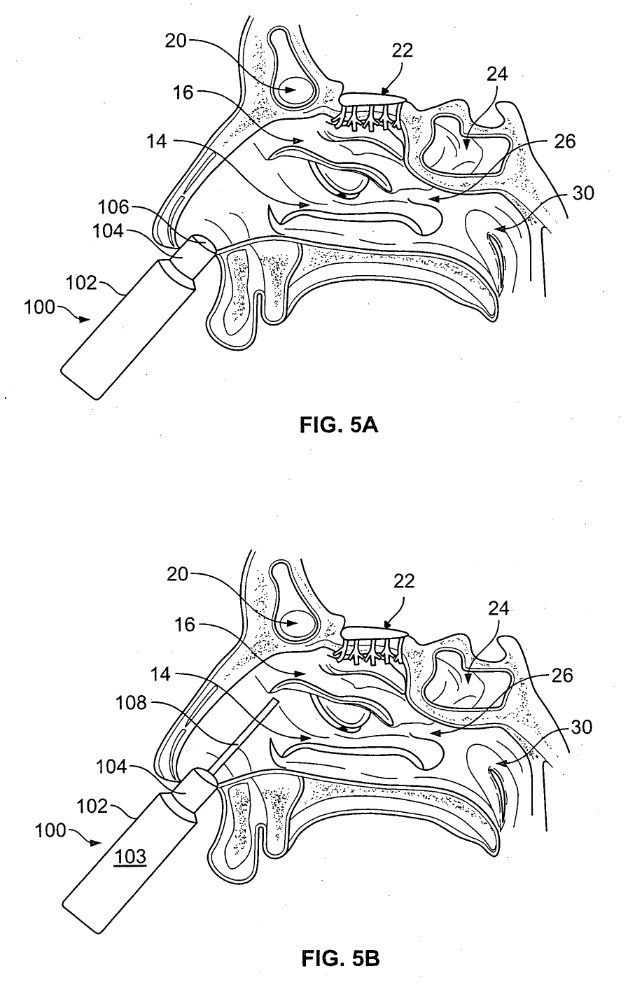

Another embodiment of the device is shown in FIGS. 5A-5C. With initial reference to FIGS. 5A-5B, a nasal delivery device 100 is shown inserted into the nasal vestibule of a user. The numerical indicators earlier described with respect to FIGS. 1A-1B identify the regions of the nasal cavity. Device 100 is comprised of an outer housing 102 that defines a main body 103 and encloses a reservoir (not visible) for containing a substance to be delivered. The device also includes a nose portion 104 configured for insertion into a nostril of a human subject. Nose portion 104 includes an opening 106 through which a tubular delivery member 108, visible in FIG. 5B, can extend and retract. Delivery member 108 is in fluid communication with the reservoir via an internal passageway not visible in the drawing. Delivery member 108 includes an outlet at its distal end for release of the substance.

Disposed on the outer circumference of the delivery member is one or more structures configured to expand prior to or during actuation of the device. FIG. 5C shows an expandable structure 110 in its expanded condition, relative to a relaxed, non-expanded condition shown in FIG. 5B. The expandable structure may take a variety of configurations and may be prepared from a variety of materials, and examples are provided below. The expandable structure(s) serves to impede air flow to the lower and/or the middle turbinates, reducing flow to the cavities surrounding the lower and middle turbinates, the inferior and middle meatuses, respectively. Blocking airflow to these regions redirects the air to the upper turbinate (superior conchae) for contact with the olfactory region.

The expandable structure(s) may take a variety of configurations, and are generally referred to herein as means for impeding flow, intending that the structures serve to at least partially block air flow to either or both of the lower and middle turbinates. The means for impeding flow can be, for example, one or more inflatable members, such as a balloon or a cuff, that inflate upon actuation of the device, by, for example a user squeezing on the main body of the device, a user sniffing causing air to enter the main body of the device and flow into the expandable balloon, and the like.

The means for impending can also take the form of a structure fabricated from an absorbent material that expands upon contact with the nasal mucosa. Materials capable of expansion upon contact with fluids are widely known, and include, but are not limited to, cellulosic materials, such as rayon, cotton, wood pulp, and chemically stiffened, modified, or cross-linked cellulosic fibers; synthetic materials, such as polyester fibers, polyolefin fibers, absorbent foams, absorbent sponges, super absorbent polymers, absorbent gelling materials; formed fibers, such as capillary channel fibers and multilimbed fibers; or any equivalent material or combinations of materials, or mixtures of these. In one embodiment of the present invention, the expandable structure and the means for impending flow are prepared from semi-rigid to soft materials that accommodate different desired insertion depths and comfort levels.

While the expandable structure as depicted in FIG. 5C is disposed about the circumference of the delivery member, however, the expandable structure(s) can be disposed in selected regions of the delivery member and when more than one expandable member is present, can be contiguous or non-contiguous.

Operation of the device will now be described with continuing reference to FIGS. 5A-5C. The nosepiece 104 is inserted into one of the nasal openings of the user until the nasal opening abuts the main body of the device (FIG. 5A). The user then inhales through the nose, in many cases with the mouth closed, so that a “sniffing” action occurs. Depending on the device design, discussed above, the user may simultaneously squeeze on the body of the device. The sniffing action alone or in combination with the inward squeeze on the device causes the delivery member to extend from the nosepiece of the device (FIG. 5B). At the same time, the impeding means is actuated, either by inflation of an expandable structure or by expansion of an absorbent, expandable structure (FIG. 5C). Essentially simultaneously with actuation of the delivery member and the impending means, the substance contained in the reservoir for delivery is released through the distal exit port of the delivery member. The substance is directed to the olfactory region due to the proximity of the distal outlet on the delivery member being close to the olfactory region and due to the impeding structures that redirect the airflow toward the superior conchea. After delivery of the substance, the device is removed from the nose, with the delivery member in its extended or relaxed positions and with the expandable structures expanded or relaxed, depending on the nature of the expandable structure.

It will be appreciated that in any of the device embodiments described herein the agent to be delivered can exit the tubular member along the body of the member, in addition to or instead of at the distal tip of the tubular member. For example, one or more exit ports can be placed along the body of the tubular member. A tubular member with a channel for delivery of the agent that is separate from a channel for airflow is also contemplated.

The tubular member in the exemplary devices above was actuated primarily by airflow. A spring-loaded tubular member, or a spring-loaded actuator causing extension of the tubular member, is also contemplated.

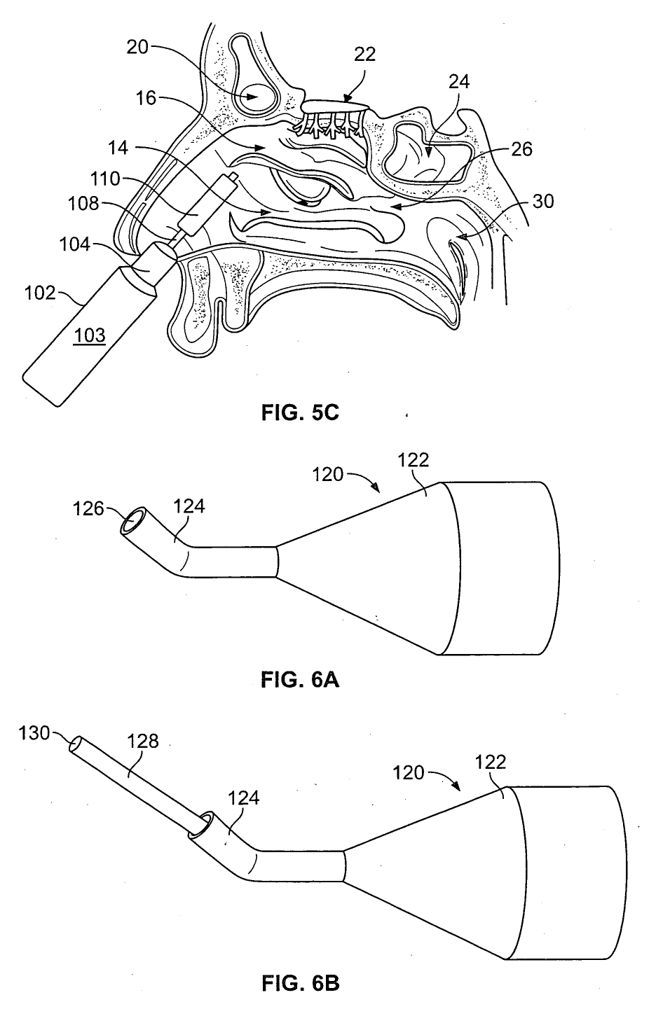

An exemplary nasal delivery device was constructed and tested. The exemplary device is shown in FIGS. 6A-6B, before and after actuation, respectively. Device 120 is comprised of a main body portion 122 and a nosepiece 124. The nosepiece and the body are integrally formed, but could be two separate units that fit together. Nosepiece 124 terminates at its distal end in an opening 126, through which a delivery member 128 extends upon actuation of the device. Delivery member is slideably contained within the nosepiece, for movement between a retracted position (FIG. 6A) and an extended position (FIG. 6B). An exit port 130 is disposed at the distal end of the delivery member, for release of a substance contained within the device.

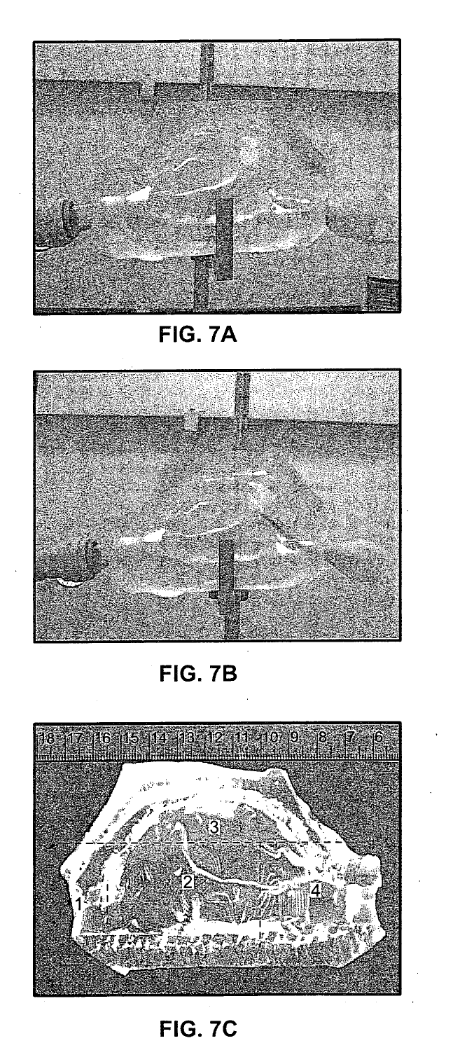

A nose model was constructed using polydimethylsiloxane, as illustrated in FIGS. 7A-7C. Openings on either end of the model served as ports for insertion of the nasal device, analogous to a nostril opening, and an exit from the nose, analogous to the nasopharynx. FIG. 7A shows the device inserted into the model nose, and FIG. 7B shows the device after actuation with the delivery member extended deep into the model nose. For analysis, the model nose was sectioned, as illustrated in FIG. 7C, where section 1 corresponds to the nostril opening, section 2 to the lower and middle turbinates, section 3 to the olfactory region, section 4 to the nasopharynx.

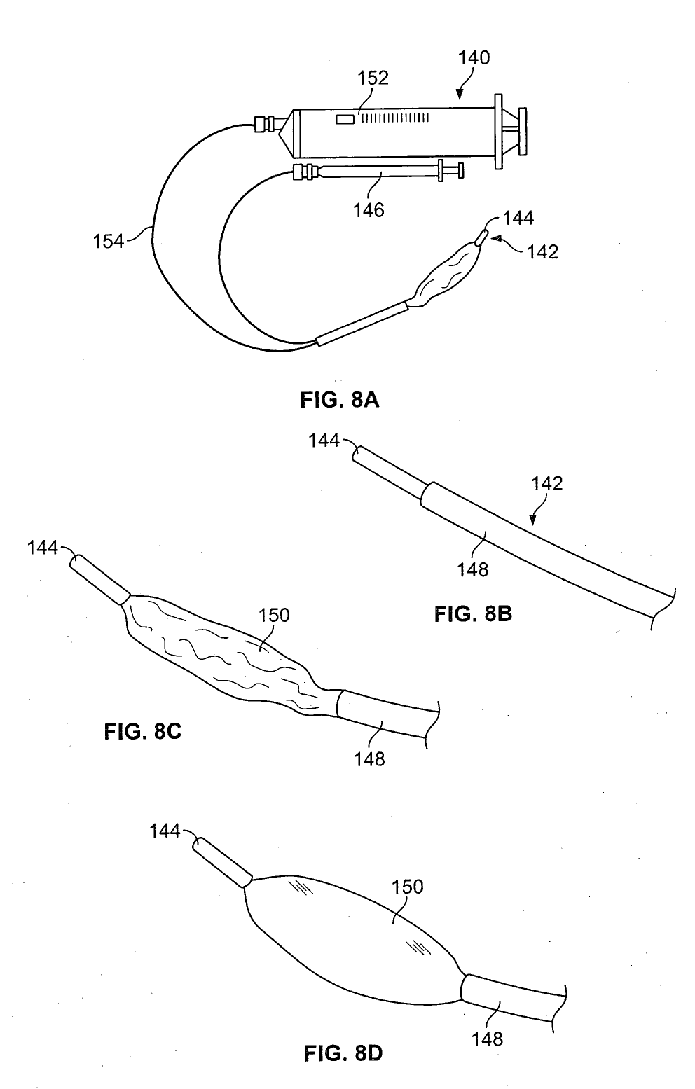

A study was conducted using the model nose of FIG. 7C and a prototype device shown in FIGS. 8A-8D. Device 140 shown in the photomicrographs of FIGS. 8A-8D includes a nozzle 142 for insertion into the model nose, the distal end of the nozzle having an outlet 144 for release of a substance contained in the drug reservoir 146. A sheath 148 is carried on the external circumference of the nozzle, the nozzle slidably disposed within the sheath (FIG. 8B). In use, the sheath is pulled back, in a proximal direction toward the device housing or drug reservoir and/or the nozzle extends from the sheath for penetration into a nasal cavity. Disposed between the sheath and the external circumference of the nozzle is an expandable structure 150, as seen in FIG. 8C. Expandable structure 150, in this embodiment, is a polymer member capable of being filled with and retaining a fluid, such as a gas or a liquid, typically air or saline. A fluid reservoir 152 is in communication via channel 154 with the expandable structure and serves to expand the structure during use, as shown in FIG. 8D. Expansion of the structure when the device is in place in the nasal cavity of a user serves to impede airflow to the lower and middle turbinates, directing air flow to the olfactory region.

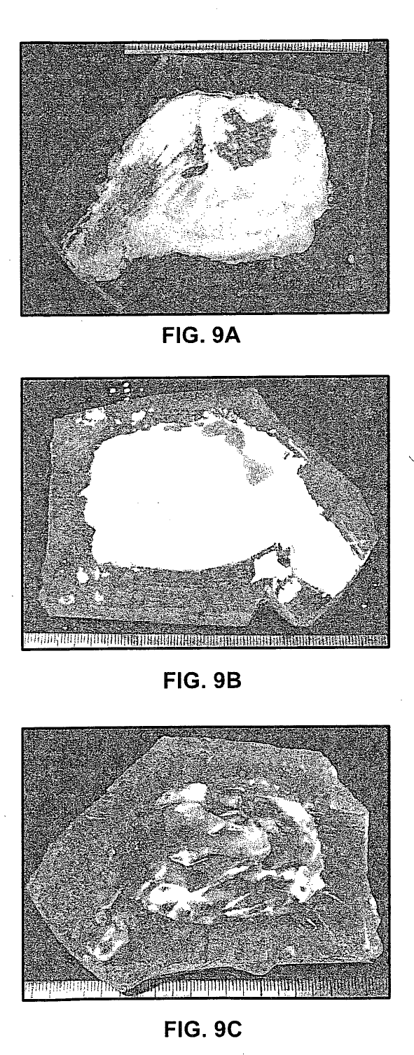

The ability of device 140 to direct flow, and thus delivery of a desired substance, to the olfactory region is shown in the photomicrographs in FIGS. 9A-9C. The photomicrographs are of model nose cavities after delivery of liquid from a commercially available 12-hour decongestant spray bottle (FIG. 9A), the prototype of FIGS. 8A-8D (FIG. 9B), and a commercially available nebulizer (FIG. 9C). The conventional spray bottle resulted in a liquid distribution pattern primarily in the nostril and the lower and middle turbinate regions (FIG. 9A). The nebulizer dispersed the liquid to nearly the entire nasal cavity (FIG. 9C). The device described herein provided a focused delivery of the liquid to the olfactory region, as seen. in FIG. 9B.

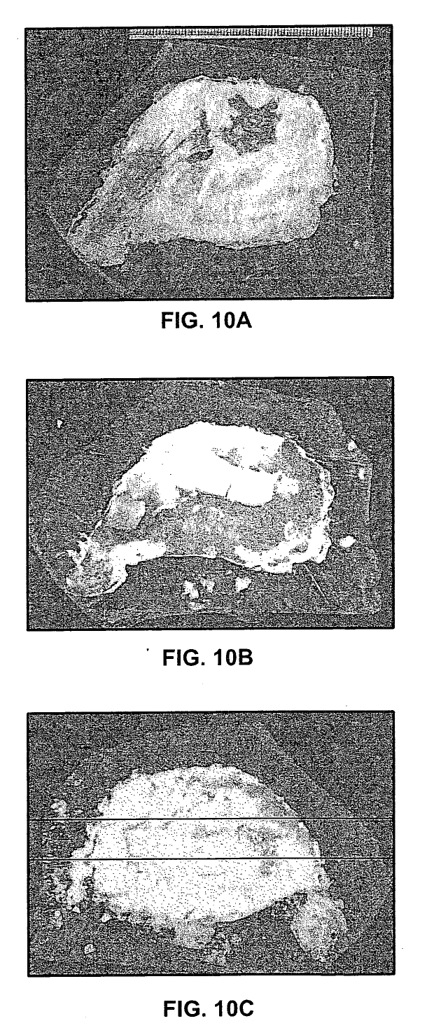

Another study was performed, using the device of FIGS. 8A-8D and the model nasal cavity and a commercially available multi-dose, non-pressurized device from Valois Corporation (VP6). A fluorescent liquid was delivered into the model nose from the devices and computer-generated photomicrographs of the model nose after delivery from the devices are shown in FIGS. 10A-10C. FIG. 10A corresponds to delivery of the liquid from the device described herein, where delivery of the substance was targeted to the olfactory region. In contrast, the commercially-available VP6 device, tested two times, resulted in delivery of the liquid to the lower and middle turbinates, with very little, if any, liquid delivered to the olfactory region (FIGS. 10B-10C). The amount of liquid dispensed to the top, middle, and lower portions of the model nose, as indicated by the lines in FIG. 10C, was quantified and is shown in Table 1.

TABLE 1
Percent of Total Dose Delivered to Regions of the Nose

| Section of Model | VP6 Commercial | Prototype |
|---|---|---|
| Nose | Device | Device |
| bottom | 12.3 | 0.9 |
| middle | 80.5 | 25.6 |
| top | 7.2 | 73.5 |

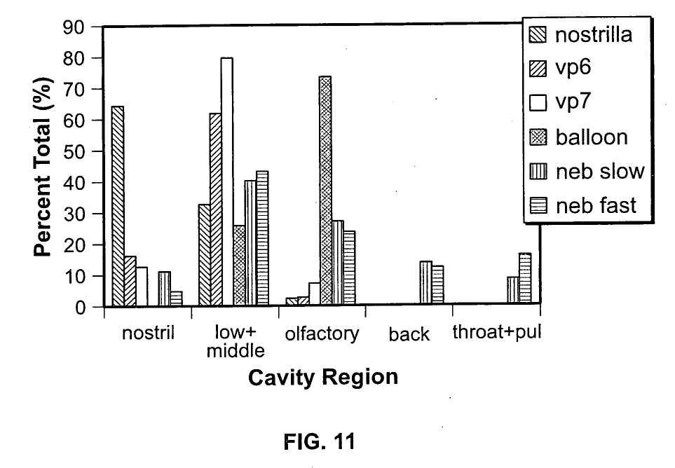

The various studies performed using the prototype device of FIGS. 8A-8D and compared to a commercially available 12-hour decongestant spray bottle (Nostrilla), multi-dose, non-pressurized devices from Valois Corporation (VP6 and VP7), a commercially available nebulizer operated at slow and fast, are summarized in FIG. 11. The amount of liquid distributed to the various regions of the model nose by each device was quantified and is shown in FIG. 11 as percent total liquid present in the nasal vestibule, the lower and middle turbinates, the olfactory region, the back of the nose cavity, and the throat and pulmonary regions. The over-the-counter decongestant spray bottle (“nostrilla”) delivered about 60% of the liquid dose to the nostril and about 30% of the dose to the lower and middle turbinates. The multi-dose devices VP6 and VP7 (“vp6” and “vp7”) delivered about 60% and about 80%, respectively, to the lower and middle turbinates. The nebulizer, operated in slow mode or fast mode (“neb slow”, “neb fast”), dispersed the dose to all the nose regions, with the largest amount of the dose (about 40%) delivered to the lower and middle turbinates. The prototype device of the invention (“balloon”) delivered about 75% of the dose to the olfactory region and about 25% to the lower and middle turbinates.

In summary, the results in FIGS. 9-11 show that the claimed device is able to target delivery of a substance being administered intranasally to the olfactory region. The device includes an extension member that penetrates into the nasal cavity to direct delivery to the olfactory region alone or in combination with a structure that acts to impede airflow to the lower and middle turbinates, thus directing more airflow to the olfactory region to increase the deposition to that region.

In another embodiment, the device additionally includes a pressure sensor effective to activate the device in response to a sniff from a user. Since sniffing increases the airflow to the olfactory region, the sniffing action in combination with the device induces the majority of the air to flow through the olfactory region, resulting in deposition of a higher amount of drugs to the target region, when compared to that achieved with existing nasal spray devices. A pressure sensor can be placed in any suitable location in the device, for example in the body housing or in the nosepiece. The pressure sensor detects the user's inhalation or “sniff” on the device by detecting the air pressure differential in the device relative to atmospheric pressure. Upon detection of a sufficient pressure differential, an activation signal can be sent to an actuating device to extend the tubular member and/or to expand the expandable structure. Any suitable pressure sensor can be used that is able to accomplish this function.

II. Methods of Use

Also provided, in another aspect, is a method delivering an active substance to the olfactory region. The method comprises providing a device substantially as described above, inserting the nosepiece of the device into a nasal vestibule, and actuating the device to deliver the active substance.

A wide variety of diseases and conditions can be treated via intranasal delivery to the olfactory region. While the methods of the present invention are useful to treat any disease or disorder that has a therapeutic target in the central nervous system (CNS), in one embodiment the methods of the present invention are useful to beneficially treat central nervous system diseases or disorders. Examples of central nervous system diseases or disorders are well known, such diseases or disorders including, but not limited to, head injury, spinal cord injury, stroke, ischemia, epilepsy, Huntington disease, Parkinson's disease, amyotrophic lateral sclerosis (ALS), Alzheimer's disease, viral encephalitis, bacterial or viral meningitis, brain tumor, spinal cord tumor, Pelizaeus-Merzbacher disease, multiple sclerosis, leukodystrophies, post-traumatic demyelination, cerebrovascular accidents, bipolar disorder, depression, anxiety disorder, schizophrenic, migraine headache, neuropathy, acute pain, breakthrough pain, chronic pain, sleep disorders, autism, lambert-Eaton syndrome, narcolepsy, insomnia, cerebral palsy, loss of smell, and dementia. Other diseases or disorders that have a CNS component include, but are not limited to, obesity, high or low blood pressure, and pain.

The device can be used for delivery of a single dose or can be used for delivery of multiple doses. As used herein, a “dose” may be any amount of a desired agent, composition, or combination of drugs or compositions. The desired substance can be a therapeutic medicament, a diagnostic agent, or prophylactic agent.

The active substance to be administered may be any active substance that is capable of treating, detecting, or preventing diseases or disorders in the olfactory region, in the brain, or in the central nervous system. Usually, but not always, the active substance will be administered to treat, detect, or prevent diseases or disorders that have a therapeutic target in the olfactory region, in the brain, or in the central nervous system. Accordingly, the term “substance” is intended to broadly cover such substances that are capable of treating, detecting, or preventing diseases or disorders, or diseases or disorders with therapeutic targets, in the olfactory region, in the brain, or in the central nervous system. Substances that can be delivered include, but are not limited to, synthetic or natural organic pharmaceuticals, radiopharmaceuticals, vitamins, synthetic or natural peptides, proteins, antibodies, hormones, vaccines, DNA and RNA, gene manipulated micro-organisms, sugars, carbohydrates, lipids, homeopathic solutions. It will be appreciated that any of these compounds can be administered with or without additional formulation to aid in the stability or in the bioavailability of the compound.

Specific examples of active substances that may be used for medical and/or prophylactic treatment of the above-mentioned diseases affecting the olfactory region or the brain, are, e.g., antiviral substances; anti-prion substances; antibacterial substances, antineoplastic substances, antiparasitic substances, anti-inflammatory substances such as ibuprofen, indomethacin, naproxen, diclofenac, tolfenamic acid, piroxicam, and the like; antidepressant substances such as imipramine, nortriptyline, pritiptylene, and the like; antifungal substances such as miconazol, ketoconazole, amphotericin B, nystatin, mepyramin, econazol, fluconazol, mycostatin, and the like. The active substance to be administered may also act as a neurotransmitter, neuromodulators, hormone, hormone releasing factor, hormone receptor agonist or antagonist, and neurotrophic factor. The active substance may also be an activator or inhibitor of a specific enzyme, an antioxidant, a free radical scavenger, a metal chelating agent, or an agent that alters the activity of ion channels of brain cell membranes, for example nimodipine. The active substance may further be any substance which is capable of acting as a stimulant, sedative, hypnotic, analgesic, anticonvulsant, antiemetic, anxiolytic, tranquillizer, cognition enhancer, agents preventing or healing amnesia, metabolic stimulator or inhibitor, appetite stimulator or inhibitor and/or narcotic antagonist or agonist. Moreover, the active substance may be any substance found to be deficient in conjunction with the brain disorder being treated or prevented, for example, nutrients such as glucose, ketone bodies, and the like, or metabolic precursors such as lecithin, choline or acetyl coenzyme A for producing neurotransmitters for the treatment of Alzheimer's disease or insulin for the treatment of obesity. The active substance may also be an antibody suitable for the treatment of viral, bacterial, prion, parasitic infections or tumors and/or cancer or for diagnosis of brain diseases or disorders where polyclonal or monoclonal antibodies and/or/with biochemical markers characteristic of the diseases or disorder are used. Such diagnostic antibodies may be labeled with any suitable labeling agent. Gene manipulated microorganisms may also be used for the treatment of tumors and/or cancer in the olfactory region or the brain.

Furthermore, in some situations the conchae (turbinates) may be enlarged, especially in allergic rhinitis. In order to pretreat or treat this enlarged conchae, especially conchae nasalis media, the enlarged conchae may be treated with an active substance having adrenergic, antihistamine, or corticosteroid effect such as ephedrin, metaoxedrin, naphazolin, tetrahydrozolin, oxymetazolin, xylometazolin, budesonid, flunisolid, beclometasonidipropionat, cocaine, or the like, in accordance with the method disclosed herein.

The active substance may be applied alone (in which case it alone constitutes the pharmaceutical preparation) or in combination with other substances. Thus, if the active substance has the appropriate physico-chemical properties the active substance may be administered alone. This may be the case where the active substances is a liquid or is in the form of a powdered substance. In many cases, however, the active substance is preferably present in a pharmaceutical preparation together with other constituents.

Pharmaceutical preparations which are suitable for use in the method according to the invention may be in the form of fluid, semisolid, or solid preparations such as powder, including microencapsulated powder, granulates, microspheres and nanospheres; liquids including solutions, dispersions, emulsions and suspensions; liposomes, gel, hydrogels, foam, ointment or gas. Preferably, however, the preparation is a liquid preparation, preferably an aqueous solution.

The optimal concentration of the active substance delivered to the olfactory region will necessarily depend upon the specific active substance used, the characteristics of the patient, and the nature of the disease or condition for which treatment is to be used. In general, a therapeutic amount of the active substance is delivered to the olfactory region. For single unit dose administration to the olfactory cavity, the volume administered is typically about 300 microliter or less per nostril, preferably about 200 microliter or less per nostril, more particularly about 100 microliter or less per nostril.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A device for delivery of a substance to the olfactory region, comprising:
a nosepiece adapted to form a substantially gas-tight seal with a nostril;
an elongate tubular member slidably disposed within the nosepiece for movement between a retracted position and an extended position, said member having (i) at a distal end an outlet, (ii) at a proximal end a means for retaining said member within said nosepiece when said member is in its extended position, and (iii) a delivery channel extending from the distal end to the proximal end, said elongate member adapted for movement in response to fluid flow from said proximal end to said distal end; and
a reservoir in flow communication with said elongate tubular member, said reservoir containing the substance to be delivered.

2. The device of claim 1, wherein said means for retaining comprises a detent.

3. The device of claim 1, wherein said a means for retaining comprises a difference in diameter of said tubular member between the proximal and distal ends.

4. The device of claim 1, wherein said elongate tubular member further includes means for impeding flow.

5. The device of claim 4, wherein said means for impeding flow comprises an absorbent member disposed on said tubular member.

6. The device of claim 4, wherein said means for impeding flow comprises one or more inflatable regions disposed on said tubular member.

7. The device of claim 6, wherein said elongate tubular member includes a fluid passageway in flow communication with said one or more inflatable regions.

8. The device of claim 7, wherein said delivery channel and said fluid passageway are coaxial.

9. The device of claim 7, wherein said delivery channel and said passageway are adjacent lumens.

10. The device of claim 1, further including a pressure sensor.

11. The device of claim 10, wherein the pressure sensor activates in response to a change in airflow to the olfactory region.

12. The device of claim 10, wherein said pressure sensor is positioned in said nosepiece or in said delivery channel.

13. The device of claim 1, further including a flow control valve.

14. The device of claim 13, wherein said valve is disposed between said nosepiece and said reservoir.

15. A method for delivering an active substance to the olfactory region, comprising:
providing a device according to claim 1;
inserting the nosepiece into a nasal vestibule; and
actuating the device to deliver the active substance.

16. The method of claim 15, wherein the active substance is for treating a disease or disorder having a therapeutic target in the central nervous system.

17. The method of claim 16, wherein the active substance is for treating a central nervous system disease or disorder.

18. The method of claim 15, wherein the active substance is an organic compound.

19. The method of claim 15, wherein the active substance is a peptide or protein.
